(19) United States
(12) Patent Application Publication
(10) Pub. No.: US 2006/0029600 A1
(43) Pub. Date: Feb. 9, 2006

(54) Treatment of viral encephalitis by agents blocking alpha-VLA-4 integrin function

(75) Inventors: Steven A. Rubin, Frederick, MD (US); Theodore A. Yednock, Forest Knolls, CA (US); Kathryn M. Carbone, Adams Town, MD (US)

Correspondence Address: BUCHANAN INGERSOLL LLP;(INCLUDING BURNS, DOANE, SWECKER & MATHIS), 12230 EL CAMINO REAL, SUITE 300, SAN DIEGO, CA, 92130, US

(73) Assignees: Elan Pharmaceuticals, Inc., San Diego, CA (US); Athena Neurosciences Johns Hopkins University

(21) Appl. No.: 11/016,386

(22) Filed: Dec. 16, 2004

Related U.S. Application Data
(63) Continuation of application No. 09/010,377, filed on Jan. 21, 1998, now abandoned.

(51) Int. Cl. A61K 39/395

(52) U.S. Cl. 424/142.1

(57) ABSTRACT

The invention provides methods of treating viral encephalitis in a patient. Such methods entail administering to the patient an effect amount of an agent that inhibits binding of leukocytes to brain endothelial cells via leukocyte surface antigen alpha-4 integrin. Such agents include antibodies and small molecules that specially bind to alpha-4 integrin.

### GOVERNMENT INTEREST

The work described in this application was supported, in part, by National Institutes of Health Grant Nos. NS289599 and MIH 48948. The U.S. Government may have certain rights in this invention.

### BACKGROUND OF THE INVENTION

A large number of viruses, including herpes viruses and arboviruses, cause encephalitis concurrent with and/or subsequent to active infection. Acute viral encephalitis viruses commonly occurs in childhood, particularly in the first 6 months of life with an incidence of one in 500-1000 infants. Arboviruses are a source of epidemics that can affect all ages, particularly in the far East.

In general, the outcome of a viral infection depends greatly on the efficiency and the speed of the immune system's reaction to the viral agent. The immune system is designed for efficient and rapid elimination of viruses to avoid spread of infection and to reduce tissue destruction, and many CNS viral infections are cleared from the brain by the immune system response. The immune reaction can however have considerable deleterious effects on the host. Particularly, for viruses that are poorly or noncytopathic, the immune response may be excessive and cause damage substantially greater than resulting from the underlying infection. One manifestation of such damage is the development of inflammation in the brain, referred to as encephalitis.

The migration of lymphocytes from the peripheral blood across the blood brain barrier to the site of encephalitis has been reported to initiate development of several central nervous system (CNS) inflammatory diseases. Studies using experimental allergic encephalomyelitides (EAE), an experimentally induced demyelinating disease of the CNS and lymphocytic choriomeningitis virus (LCMV) infection models report that T-lymphocyte entry into the CNS is mediated by cellular adhesion molecules. See O'Neill et al., Immunology 72:520-525 (1991); Raine et al., Lab. Invest. 63:476-489 (1990); Yednock et al., Nature 356:63-66 (1992); Baron et al., J. Exp. Med. 177:57-68 (1993); Steffen et al., Am. J. Path 145:189-201 (1994); Christensen et al., J. Immunol. 154:5293-5301 (1995).

Cellular adhesion molecules are cell surface molecules involved in the direct binding of one cell to another (Long et al., Exp. Hematol 20:288-301 (1992)). The integrin and the immunoglobulin super gene families of adhesion molecules have been shown to be key in CNS lymphocyte trafficking (Hemler et al., Annu. Rev. Immunol. 8:365-400 (1990); Springer et al., Cell 76:301-314 (1994); Issekutz et al., Curr. Opin. in Immunol. 4:287-293 (1992)). The integrin group of adhesion molecules are heterodimers composed of non-covalently linked A and B chains (Hemler et al., Annu. Rev. Immunol. 8:365-400 (1990)). There are multiple families of integrins, members of which share a common B chain. A receptor present on the surface of most circulating T-lymphocytes is α4β1 integrin (VLA-4). This integrin has two counterreceptors on endothelial cells, vascular cell adhesion molecule (VCAM-1) and fibronectin. (Elices et al., Cell 60, 577-584 (1990)). VCAM-1 is a member of the immunoglobulin supergene family present on the surface of endothelial cells (Elices et al., Cell 60:577-584 (1990); Carlos et al., Blood 76:965-970 (1990); Shimizu et al., Immunol. Today 13:106-112 (1992)). Several studies have shown that VLA-4 and, in particular the α4 integrin subunit, plays a prominent role during inflammation of the CNS (Yednock et al., Nature 356:63-66 (1992); Baron et al., J. Exp. Med. 177:57-68 (1993); Steffen et al., Am. J. Path 145:189-201 (1994);

Christensen et al., supra. It has also been reported that VCAM-1 expression is elevated in inflamed brain tissue relative to normal brain tissue. See Cannella & Raine, Ann. Neurol. 37, 424-435 (1995); Washington et al., Ann. Neurol. 35, 89-97 (1994); Dore-Duffy et al., Frontiers in Cerebral Vascular Biology: Transport and Its Regulation, 243-248 (Eds. Drewes & Betz, Plenum, N.Y. 1993)

The up-regulation of cellular adhesion molecule expression on endothelium during EAE or LCMV infection in vivo and the ability of anti-VLA-4 antibodies to prevent the development of inflammation in these models has led to the following proposed model (Christensen et al., supra; Osborn et al., Cell 62:3-6 (1990); Cannella et al., Lab. Invest. 65:23-31 (1991); Yednock et al., nature 356:63-66 (1992); Baron et al., J. Exp. Med. 177:57-68 (1993). Antigen-primed T-lymphocytes randomly leave the circulation and enter the CNS, where, by chance, they encounter their specific antigen. This interaction leads to a release of cytokines from T-lymphocytes resulting in the up-regulation of appropriate adhesion molecules, thereby recruiting effector cells and more lymphocytes to the local area (Baron et al., supra; Christensen et al., supra. Although the majority of recruited cells are nonspecific, some cells are responsive to antigens presented at the inflammatory site. Thus, nonactivated T-lymphocyte infiltrates in CNS tissue are naive cells (Cross et al., Lab. Invest. 63:162-170 (1990); Wekerle et al., TINS 9:271-277 (1986); Wekerle et al., J. Exp. Biol. 132:43-57 (1987); Hickey et al., J. Neurosci. Res. 28:254-260 (1991)).

Borna disease virus (BDV) serves as a model for viral encephalitic infections. BDV, an 8.9 kb negative strand RNA virus, produces sporadic but fatal neurological disease in horses and sheep (Rott et al., Springer-Verlag 17-30 (1995)). Experimentally, BDV persistently infects a broad spectrum of species ranging from chickens to primates, and possibly, humans (Waltrip et al., Psychiatry Res. 56:33-44 (1995); Bode et al., Nature Med. 1:232-236 (1995); Kishi et al., FEBS Lett 15:293-297 (1995)). Like EAE and LCMV, borna disease virus (BDV) causes a severe T-lymphocyte mediated meningoencephalitic response in the brain (Sitz et al., Springer-Verlag 75-92 (1995)). For the most part, Borna disease is due to the immune response to BDV antigens, rather than direct effects of BDV damage to the brain. As in other forms of CNS inflammation, it has been found that activated, BDV-antigen specific, T-lymphocytes express α4 integrin (Plaz et al., J. Virol. 69:896-903 (1995)). The course of BDV infection illustrates the complex role of inflammatory mechanisms in viral encephalitis. It has been found that BDV-specific CD4+ T-cells can both prevent and augment Borna disease depending on the stage of infection. When administered to an experimental animal before infection, the cells are protective. When administered after infection, they augment symptoms of disease. See Richt et al., J. Exp. Med. 179, 1467-1473 (1994).

In view of the complex role of inflammation in viral encephalitis, it was unpredictable at which therapeutic target attempts to abort inflammation should best be directed, and whether such attempts would ameliorate or exacerbate this disease. Notwithstanding these uncertainties and difficulties, the present invention provides inter alia methods of treating viral encephalitis employing therapeutic agents that block binding of alpha-4 integrin to brain endothelial cells.

### BRIEF DESCRIPTION OF THE FIGURES

FIG. 1: Severity of Borna disease rated on a 0 to 4+ scale in BDV-infected rats (open bars) and BDV-infected/MAb treated rats (hatched bars) on days 26 and 30 post BDV-inoculation. *p<0.05.

FIG. 2: Mean weights (g) of uninfected (black bars), BDV-infected (hatched bars) and BDV-infected/MAb treated (open bars) rats on days 26 and 30 post BDV-inoculation. *p<0.05.

FIG. 3: Reduction in inflammatory responses to BDV in brain from rats treated with an anti-alpha-4 integrin monoclonal antibody (day 30 post BDV-inoculation). (A) BDV-infected rat brain showing extensive perivascular cuffing (arrow);(B) BDV-infected rat brain showing a reduction in perivascular cuffing following anti-alpha-4 integrin monoclonal antibody treatment (arrow); (C) uninfected rat brain control without encephalitis (arrows). Hematoxylin and eosin stain; magnification, ×200.

### DEFINITIONS

Specific binding between an antibody or other binding agent and apha-4 integrin or VCAM-1 means a binding affinity of at least 10⁶ M⁻¹. Preferred binding agents bind with affinities of at least about 10⁷ M⁻¹, and preferably 10⁸ M⁻¹ to 10⁹ M⁻¹ or 10¹⁰ M⁻¹.

The term epitope means a protein determinant capable of specific binding to an antibody. Epitopes usually consist of chemically active surface groupings of molecules such as amino acids or sugar side chains and usually have specific three dimensional structural characteristics, as well as specific charge characteristics.

The term antibody is used to mean whole antibodies and binding fragments thereof.

Unless otherwise indicated patient refers to a human patient. A pediatric patient is a patient up to two years old.

### SUMMARY OF THE CLAIMED INVENTION

The invention provides methods of treating viral encephalitis in a patient. Such methods entail administering to the patient an effect amount of an agent that inhibits binding of leukocytes to brain endothelial cells via leukocyte surface antigen alpha-4 integrin. Agents can be administered to patients before or after viral infection. Agents can also be administered whether or not a patient is currently exhibiting symptoms of encephalitis. In some methods, the patient is infected with a herpes virus or an arbovirus. In some methods, the patient is monitored for symptoms of encephalitis. In some methods, the agent specifically binds to alpha-4 integrin as a subunit of VLA-4. Agents include antibodies and small molecules. Some agents bind to an epitope of alpha-4 integrin formed by association with alpha-1 integrin in VLA-4, which epitope is not present in other complexes containing alpha-4, such as alpha-4 beta-7 complex. In some methods, an agent of the invention is administered in combination with an antiviral agent or another antiinflammatory agent. In some methods, the agent is formulated with a carrier as a pharmaceutical composition. In some methods, the patient is a pediatric patient.

### DETAILED DESCRIPTION

I. Therapeutic Agents

A. Binding Specificity and Functional Properties

Therapeutic agents of the invention function by inhibiting or preventing leukocytes bearing alpha-4 integrin (a subunit of VLA-4) from binding to endothelial cells of the CNS systems, thereby aborting the inflammatory process. Many of the therapeutic agents function by specifically binding to an epitope of the alpha-4 integrin subunit required for interaction with VCAM-1, thereby competing with VCAM-1 for binding to alpha-4 integrin and reducing or eliminating binding of alpha-4 integrin to VCAM-1. Some therapeutic agents of the invention bind to an epitope of alpha-4 integrin that is present when alpha-4 is associated with beta-1 in VLA-4 but absent when alpha-4 is associated with other subunits (e.g., α4β7). An antibody having this specificity is described by Bednarczyk et al., J. Biol. Chem. 269, 8348-8354 (1994). Other therapeutic agents specifically bind to brain endothelial receptors, particular, VCAM-1, that interact with alpha-4 integrin in producing an inflammatory response. For example, some therapeutic agents specifically bind to an epitope of VCAM-1 that interacts with alpha-4 integrin thereby competing with alpha-4 integrin for binding to VCAM-1 and reducing or eliminating binding between VCAM-1 and alpha-4 integrin. Other therapeutic agents function by suppressing expression of alpha-4 integrin or VCAM-1.

Potential therapeutic agents are tested for appropriate binding specificity by a variety of assays. These include a simple binding assay for detecting the existence or strength of binding of an agent to cells bearing alpha-4 integrin or VCAM-1. The subset of agents binding to an alpha-4 epitope formed by association with beta-1 subunit in VLA-4 can then be identified, if desired, by screening antibodies for lack of binding to a cell line expressing alpha-4 in a complex other than alpha-4 beta-1. For example, a cell line expressing alpha-4 beta-7 disclosed by Hemler, Immunological Reviews 114, 45-65 (1990) is suitable.

The agents are also tested for their capacity to block the interaction of VLA-4 receptor with inflamed endothelial cells, other cells bearing a VCAM-1 counterreceptor, or purified VCAM-1 counterreceptor. Usually, the assay is performed with VLA-4 and VCAM-1 expressed on the surface of cells. For example, a Ramos cell line expressing VLA-4 and VCAM-1 transfected L-cells are suitable. Endothelial cells bearing VCAM-1 can be grown and stimulated in culture or can be a component of naturally occurring brain tissue sections. See Rubin et al., WO 91/05038. Rubin et al. further describe a blood-brain barrier model for use in screening assay. The barrier is formed from brain endothelial cells bearing VCAM-1 immobilized to a support. Appropriate blocking activity of an agent can be confirmed by in vivo testing on an experimental animal, such as a mouse or rat, infected with Borna disease virus, as discussed in the Examples.

B. Existing Therapeutic Agents

A number of therapeutic agents suitable for use in the present methods are already available. Monoclonal antibodies to the alpha-4 subunit of VLA-4 that block binding to VCAM-1 include HP2/1 (AMAC, Inc. Westbrook Me., Product #0764), L25 (Clayberger et al., J. Immunol. 138, 1510 (1987)), TY 21.6 (WO 95/19790), TY.12 -(Rubin et al., supra) and HP2/4. Further antibodies binding to VLA-4 and blocking VCAM-1 binding are described by Biogen, WO 94/17828. Humanized antibodies to alpha-4 integrin are described by Athena Neurosciences, WO 95/19790. Preferred humanized antibodies are derived from the mouse 21.6 antibody. An exemplified antibody has a light chain variable domain comprising SEQ. ID. No. 1 and a heavy chain variable domain comprising sequence ID. No. 2.

Athena Neurosciences, WO 96/01644 discloses peptides that inhibit binding of VLA-4 to VCAM-1. The peptides have a binding affinity for VLA-4 with an IC50 of 50 μM or less. The peptides have the formula (R1-Y/F-G/E-R2)n or R-PVSF-R′ (II). R and R′ are sequences of 0-7 amino acid totalling not more than 9 amino acids. R1 is a sequence of 0-6 amino acids and R2 is a sequence of 1-7 amino acids, totalling not more than 2-11 amino acids. N is 1 or 2. Optionally 1 amino acid is a D-amino acid and the N terminus is optionally modified by attachment of R4-CO— or R5-O. The C terminus is optionally modified by replacement of OH by NR7R8 or O—R6; R4=H, lower alkyl, cycloalkyl, aryl or aralkyl. R5 is as R4 but not H. R6 is as R5. R7 and R8 are as R4. Other peptides, peptide derivatives or cyclic peptides that bind to VLA-4 and block its binding to VCAM-1 are described by Biogen, WO 96/22966; Zeneca, WO 96/20216; Texas Biotechnology Corp., U.S. Pat. No. 5,510,332; Texas Biotechnology Corp, WO 96/00581; Cytel, WO 96/06108.

Monoclonal antibodies that bind to VCAM-1 and block its interaction with VLA-4 are described by e.g., Hadasit Medical Res. Services & Dev, WO 95/30439. Other antibodies to VCAM-1 have been reported by Carlos et al., Blood 76, 965-970 (1990) and Dore-Duffy et al., Frontiers in Cerebral Vascular Biology: Transport and Its Regulation, pp. 243-248 (Eds. Drewes & Betz, Plenum, N.Y. 1993). Small molecules that bind to VCAM-1 and inhibit its interaction with VLA-4 are also know. See Warner Lambert, WO 96/31206 (describing flavones and coumarins).

Other suitable agents act by suppressing VCAM-1 expression thereby inhibiting leukocytes bearing VLA-4 from binding to CNS endothelial cells. Sandoz, WO 96/03430 and Emory University, U.S. Pat. No. 5,380,747 respectively describes cyclopeptolides and dithiocarbamates for suppressing expressing of VCAM-1.

C. Production of Additional Therapeutic Agents

1. Antibodies

a. General Characteristics

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7 (incorporated by reference in its entirety for all purposes).

The variable regions of each light/heavy chain pair form the antibody binding site. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. CDR and FR residues are delineated according to the standard sequence definition of Kabat et al., supra. An alternative structural definition has been proposed by Chothia et al., J. Mol. Biol. 196, 901-917 (1987); Nature 342, 878-883 (1989); and J. Mol. Biol. 186, 651-663 (1989).

b. Production

Antibodies to alpha-4 integrin or VCAM-1 can be produced by a variety of means. The production of non-human monoclonal antibodies, e.g., murine or rat, can be accomplished by, for example, immunizing the animal with cells expressing VCAM-1, VLA-4 or the alpha-4 subunit thereof, or a purified preparation of one of these receptors or a fragment thereof. Such an immunogen can be obtained from a natural source, by peptides synthesis or by recombinant expression. Both VLA-4 and VCAM-1 have been cloned and expressed (Hemler, EP 330,506; Osborne et al., Cell 59, 1203-1211 (1989)). Therefore, in general, production of antibodies to these molecules presents no particular difficulties. Polyclonal antibodies can be obtained from serum of the animal. Alternatively, antibody-producing cells obtained from the immunized animals are immortalized and screened for the production of an antibody which the binding specificity described above. See Harlow & Lane, Antibodies, A Laboratory Manual (CSHP NY, 1988) (incorporated by reference for all purposes). Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861 (incorporated by reference for all purposes).

Human antibodies can be obtained using phage-display methods. See, e.g., Dower et al., WO 91/17271; McCafferty et al., WO 92/01047. In these methods, libraries of phage are produced in which members display different antibodies on their outersurfaces. Antibodies are usually displayed as Fv or Fab fragments. Phage displaying antibodies with a desired specificity are selected by affinity enrichment to VCAM-1 or alpha-4 integrin, or fragments thereof. Human antibodies can be selected by competitive binding experiments, or otherwise, to have the same epitope specificity as a particular mouse antibody, Such antibodies are particularly likely to share the useful functional properties of the mouse antibodies.

c. Antibody Fragments

Typically, fragments compete with the intact antibody from which they were derived for specific binding to alpha-4 integrin or VCAM-1 and bind with an affinity of at least 10⁶, 10⁷, 10⁸, 10⁹ M⁻¹, or 10¹⁰ M⁻¹. Antibody fragments include separate heavy chains, light chains Fab, Fab° F(ab′)₂, Fv, and single chain antibodies comprises a heavy chain variable region linked to a light chain variable region via a peptide spacer. Fragments can be produced by enzymic or chemical separation of intact immunoglobulins. For example, a F(ab′)₂ fragment can be obtained from an IgG molecule by proteolytic digestion with pepsin at pH 3.0-3.5 using standard methods such as those described in Harlow and Lane, supra. Fab fragments may be obtained from F(ab′)₂ fragments by limited reduction, or from whole antibody by digestion with papain in the presence of reducing agents. (See id.) Fragments can also be produced by recombinant DNA techniques. Segments of nucleic acids encoding selected fragments are produced by digestion of full-length coding sequences with restriction enzymes, or by de novo synthesis. Often fragments are expressed in the form of phage-coat fusion proteins. This manner of expression is advantageous for affinity-sharpening of antibodies.

d. Recombinant Expression of Antibodies

Nucleic acids encoding light and heavy chain variable regions, optionally linked to constant regions, are inserted into expression vectors. The light and heavy chains can be cloned in the same or different expression vectors. The DNA segments encoding antibody chains are operably linked to control sequences in the expression vector(s) that ensure the expression of antibody chains. Such control sequences include a signal sequence, a promoter, an enhancer, and a transcription termination sequence. Expression vectors are typically replicable in the host organisms either as episomes or as an integral part of the host chromosome. Suitable hosts include E. coli, yeast, and mammalian cells.

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (see generally Scopes, Protein Purification (Springer-Verlag, N.Y., 1982). Substantially pure immunoglobulins of at least about 90 to 95% homogeneity are preferred, and 98 to 99% or more homogeneity most preferred.

Many of the antibodies described above can undergo non-critical amino-acid substitutions, additions or deletions in both the variable and constant regions without loss of binding specificity or effector functions, or intolerable reduction of binding affinity (i.e., below about 10⁶ M⁻¹) for alpha-4 integrin or VCAM-1. Preferred antibody light and heavy chain sequence variants have the same complementarity determining regions (CDRs) as the corresponding chains from one of the above reference antibodies. Occasionally, a mutated immunoglobulin can be selected having the same specificity and increased affinity compared with a reference immunoglobulin from which it was derived. Phage-display technology offers powerful techniques for selecting such immunoglobulins. See, e.g., Dower et al., WO 91/17271 McCafferty et al., WO 92/01047; Huse, WO 92/06204.

2. Other Therapeutic Agents

Other therapeutic agents that block binding of the alpha-4 integrin to activated brain endothelial cells can be obtained by producing and screening large combinatorial libraries. Combinatorial libraries can be produced for many types of compound that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes). Peptide libraries can also be generated by phage display methods. See, e.g., Devlin, WO 91/18980. The libraries of compounds can be initially screened for specific binding to the alpha-4 integrin subunit of VLA-4 or to VCAM-1, optionally in competition with a reference compound known to have blocking activity. Appropriate activity can then be confirmed using one of the assays described above.

II. Viruses Causing Encephalitis

Classes of viruses causing encehpalitis include bunyaviridae, flaviviridae, togaviridae, reoviridae, picornaviridae, rhabdoviridae, herpesviridae, retroviridae, orthomyxoviridae, papovaviridae, arenaviridae, and paramyxoviridae. Examples of specific human pathogens include California encephalitis virus, LaCrosse virus (bunyaviridae), St. Louis encephalitis virus (flaviviridae), Eastern and Western equine encephalitis virus (togaviridae), Colorado tick fever virus (reoviridae), coxsackie viruses, enteroviruses, polioviruses (picornaviridae), rabies (rhabdoviridae), herpes simplex virus, varicella zoster virus (herpesviridae), human immunodeficiency viruses (retroviridae), influenza viruses (orthomyxoviridae), JC virus (papovaviridae), lymphocytic choriomeningitis virus (arenaviridae), mumps and measels (Paramyxoviridae), and Borna disease virus.

HSV-I is the most common cause of sporadic fatal encephalitis in the Eastern World. See Whitely & Lakeman, Clin Infect. Dis. 20, 414-420 (1995). Both primary and recurrent HSV infections can result in herpes simplex encephalitis (HSE). Clinical presentations of HSE range from a mild illness to diffuse cerebral disease and focal necrotizing lesions. (13, 14, 15. HSV-II also causes CNS infections, which can result in fulminant encephalitis, especially in the neonatal period, or milder meningitis. Epstein Barr Virus (EBV) can cause a variety of neurological disturbances including meningitis, polyneuritis, encephalomyelitis and mononeuritis. Varicella zoster virus has been reported to cause encephalitis in immunosuppressed adults. Human herpesvirus 6, which causes Roseola infantum, has been associated with serious neurological complications, such as miningoencephalitis, status epilepticus, transverse myelitis and recurrent febrile seizures. Human herpesvirus 7, another cause of roseola infantum, has been associated with infantile hemiplegia. Cytomegalovirus (CMV) causes congenital infection, which may have CNS complications. Further, in immunocompromised patients, CMV causes more severe neurological illness.

The enterovirus group of RNA viruses include poliovirus, coxsackievirus, echovirus and the numbered enteroviruses. These viruses cause a number of CNS complications including septic meningitis and encephalitis. Rotbart, Clin. Infect. Dis. 20, 971-981 (1995).

Arboviruses are responsible for most outbreaks of epidemic encephalitis. In the Western hemisphere the most important types are eastern and western equine, Venezuelan, St. Louis and California. Types found elsewhere include Japanese B, Murray Valley, and tichborne. All have vertebrate hosts and mosquito vectors except for the tickborne. The brain is the principal site of infection. Infection causes seizures, confusion, delirium or coma.

HIV-1 also infects the CNS. At least four syndromes have been ascribed to the direct effects of the virus including an acute aseptic meningitis or more rarely, encephalitis, a subacute encephalitis, a vacuolar myelopathy and a peripheral neuropathy. Subacute encephalitis is characterized pathologically by microscopic foci of multinucleated giant cells, macrophages and lymphocytes together with microglial cells, reactive astrocytes and some vacuolation and pallor of the surrounding myelin.

Some viruses, such as Semliki forest virus, are used in combination with injections of spinal cord homogenate and radiation to induce experimental alerigic encephalomyelitis (EAE) in laboratory animals, a syndrome that simulates multiple sclerosis in humans. See Hanninen et al., J. Neuroimmunol. 72, 95-105 (1997). Multiple sclerosis is a complex autoimmune syndrome, probably of multifactorial origin, in contrast to simple viral encephalitis, which is caused by an inflammatory response to viral infection. Typically, the present methods are not employed on EAE animals, or on humans suffering from multiple sclerosis.

III. Diagnosis of Encephalitis

Viral encephalitis can be acute or chronic. Acute viral encephalitis is characterized by fever, headache, decreased mentation (e.g., somnolence, sleepiness or coma), paralysis, loss of sight or hearing, and sometimes death. Chronic encephalitis is usually accompanied by less severe signs of general illness (e.g., fever, coma) and is characterized by symptoms of behavioral disease (e.g., decreased ability to think clearly, depression). The most characteristic histologic features of viral disease are a perivascular and parenchymal mononuclear cell infiltrate (lymphocytes, plasma cells and macrophages) glial nodules and neuronophagia. Intranuclear inclusion bodies are seen in many viral infections.

Diagnosis and disease monitoring are usually based on the combination of clinical assessment, exclusion of other causes and specific investigations. Investigation of the patient with suspected encephalitis may include electroencephalography, cranial computed tomography, magnetic resonance imaging and cerebrospinal imaging and culture, or PCR with primers that bind to viral sequences in the test sample. Rotbart, Clin. Infect. Dis. 20, 971-981 (1995); Tyler, Ann. Neurol. 36, 809-811 (1994); O'Meara, Current Opinion in Pediatrics 8, 11-15 (1996)).

IV. Pharmaceutical Compositions

The invention provides pharmaceutical compositions to be used for prophylactic or therapeutic treatment comprising an active therapeutic agent, e.g., an antibody, and a variety of other components. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

For parenteral administration, the therapeutic agents of the invention can be administered as injectable dosages of a solution or suspension of the substance in a physiologically acceptable diluent with a pharmaceutical carrier which can be a sterile liquid such as water and oils with or without the addition of a surfactant and other pharmaceutically preparations are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. The agents of this invention can be administered in the form of a depot injection or implant preparation which can be formulated in such a manner as to permit a sustained release of the active ingredient. A preferred composition comprises monoclonal antibody at 5 mg/mL, formulated in aqueous buffer consisting of 50 mM L-histidine, 150 mM NaCl, adjusted to pH 6.0 with Hcl.

V. Therapeutic Methods

Therapy is usually initiated on diagnosis of viral encephalitis, and continued at regular intervals (e.g., weekly) until the symptoms of encephalitis are detectably reduced, arrested or reversed. In some instances, therapy can be administered prophylactically to patients at risk of infection by a virus causing encephalitis before symptoms of encephalitis are apparent. Such patients include neonates whose mothers are infected with a virus causing encephalitis, and immunosuppressed patients (e.g., transplant, cancer or AIDS patients).

In therapeutic applications, compositions are administered to a patient suspected of, or already suffering from such a disease in an amount sufficient to cure, or at least partially arrest, the symptoms of the disease and its complications. An amount adequate to accomplish this is defined as a therapeutically- or pharmaceutically-effective dose.

In prophylactic applications, pharmaceutical compositions are administered to a patient susceptible to, or otherwise at risk of, disease in an amount sufficient to eliminate or reduce the risk or delay the outset of the disease. Such an amount is defined to be a prophylactically effective dose. Compositions may be administered to mammals for veterinary use and for clinical use in humans. Effective doses of the compositions vary depending upon many different factors, including means of administration, target site, physiological state of the patient, and other medicants administered. Thus, treatment dosages need to be titrated to optimize safety and efficacy. In general, the administration dosage will range from about 0.0001 to 100 mg/kg, and more usually 0.01 to 5 mg/kg of the host body weight.

The pharmaceutical compositions are administered by parenteral, topical, intravenous, oral, or subcutaneous, intramuscular local administration, such as by aerosol or transdermally, for prophylactic and/or therapeutic treatment. In a preferred treatment regime, the composition is administered by intravenous infusion or subcutaneous injection at a dose from 1 to 5 mg antibody per kilo of bodyweight.

Agents that block binding of alpha-4 integrin to VCAM-1 can be used with effective amounts of other therapeutic agents against acute and chronic inflammation. Such agents include antibodies and other antagonists of adhesion molecules, including other integrins, selecting, and immunoglobulin (Ig) superfamily members (see Springer, Nature 346, 425-433 (1990); Osborn, Cell 62, 3 (1990); Hynes, Cell 69, 11 (1992)). Integrins are heterodimeric transmembrane glycoproteins consisting of an α chain (120-180 kDa) and a β chain (90-110 kDa), generally having short cytoplasmic domains. For example, three important integrins, LFA-1, Mac-1 and P150,95, have different alpha subunits, designated CD11a, CD11b and CD11c, and a common beta subunit designated CD18. LFA-1 (α_{L}β₂) is expressed on lymphocytes, granulocyte and monocytes, and binds predominantly to an Ig-family member counter-receptor termed ICAM-1 and related ligands. ICAM-1 is expressed on many cells, including leukocytes and endothelial cells, and is up-regulated on vascular endothelium by cytokines such as TNF and IL-1. Mac-1 (α_{M}β₂) is distributed on neutrophils and monocytes, and also binds to ICAM-1. The third β2 integrin, P150,95 (α_{X}β₂), is also found on neutrophils and monocytes. The selectins consist of L-selectin, E-selectin and P-selectin.

Other antiinflammatory agents that can be used in combination with agents that block alpha-4 integrin binding to VCAM-1 include antibodies and other antagonists of cytokines, such as interleukins IL-1 through IL-13, tumor necrosis factors α & β, interferons α, β and γ, tumor growth factor Beta (TGF-β), colony stimulating factor (CSF) and granulocyte monocyte colony stimulating factor (GM-CSF). Other antiinflammatory agents include antibodies and other antagonists of chemokines such as MCP-1, MIP-1α, MIP-1β, rantes, exotaxin and IL-8. Other antiinflammatory agents include NSAIDS, steroids and other small molecule inhibitors of inflammation. Formulations, routes of administration and effective concentrations of agents for combined therapies are as described above for agents that block binding of alpha-4 integrin to VCAM-1.

Agents that block binding of alpha-4 integrin to VCAM-1 can also be used in combination with antiviral agents. Such agents include polyclonal sera from infected individuals and neutralizing monoclonal antibodies that bind to a virus. Other therapeutic agents abort a process in viral reproduction, such as nucleic acid replication. Examples of anti-viral agents include acyclovir, ganciclovir, famciclovir and cidofovir for treatment of herpes virus infections, such as HSV-1 and -II and CMV. Neuralizing antibodies to HSV virus are described by e.g., Su et al., J. Virol. 70, 177-81 (1996); Co et al., Proc. Natl. Acad. Sci. USA 88, 2869-73 (1991); Staats et al., J. Virol. 65, 6008-14 (1991). Other antiviral agents include ribavirin for treatment of respiratory syncytial virus (RSV), and AZT, ddI, ddC, d4T, TIBO 82150, nevaripine, 3TC, crixivan and ritonavir, which are effective in treatment of HIV.

### EXAMPLES

This study provides evidence of the usefulness of in vivo therapy with α4 integrin antibody in preventing immune mediated CNS damage following viral encephalitis.

Materials and Methods

On day 0, four week-old inbred male Lewis rats (Harlan, Indianapolis, Ind.) (n+33) were inoculated with 2×10⁴ TCID₅₀ of BDV stock (strain CRP₃), or sham inoculated (n+8) with an equal volume of uninfected material intracranially. On days 14 and 18 post infection (p.i.) one group of BDV infected rats (n=15) received an injection (intraperitoneally) of 1.0 mg of the anti-alpha-4 integrin MAb GG5/3.

On days 26 and 30 post BDV-inoculation, BDV-infected and BDV-infected/MAb-treated rats were examined for incidence and severity of Borna disease. At each time point, a representative set of three BDV-infected, five BDV-infected/MAb-treated, and two sham-inoculated rats were weighed and deeply anesthetized. The brain was removed aseptically and sagittally divided. One half of the brain was processed for viral titer by infectious focus assay as described earlier (Carbone et al., J. Virol. 61, 3431-3440 (1987)). The other half of the brain was fixed in 4% paraformaldehyde, paraffin embedded and cut into 8 micron-thick sections. To examine viral distribution in the brain, sections were stained by avidin-biotin immunohistochemistry (Vector, Burlingame, Calif.) using a polyclonal mouse anti-BDV antibody followed by biotinylated anti-mouse IgG (Vector, Burlingame, Calif.) as described previously (29). Duplicate sections were stained with hematoxylin and eosin for histological evaluation for encephalitis.

Severity of disease was assessed in a blinded fashion and ranked on a 0 to 4 scale as follows: (0) no disease, (1+) early evidence of disease (lack of grooming, increased activity), (2+) definite hyperactivity, (3+) signs of neurologic disease (ataxia, paresis, but mobile, eating and hydrated), (4+) severe disease (paralysis, immobile, unable to eat or drink, moribund).

Severity of encephalitis was characterized by the intensity and distribution of perivascular cuffing of encephalitic foci. Using hematoxylin and eosin stained sagittal brain sections, the encephalitic response was scored as follows: (0) normal, (1+) one to two layers of inflammatory infiltrates per perivascular cuff, focal; (2+) one to two layers if inflammatory infiltrates per perivascular cuff, widely distributed; (3+) three or more layers of inflammatory infiltrates per perivascular cuff, focal; (4+) three or more layers of inflammatory infiltrates perivascular cuff, widely distributed throughout brain.

All rat experimentation conformed to the National Research Council's Guide for the care and use of laboratory animals.

Results

Reduction in Prevalence of Clinical Borna Disease Following Anti-Alph-4 Integrin Monoclonal Antibody Treatment

By day 26 p.i., anti-alpha-4 integrin MAb treatment was associated with a reduction in clinical Borna disease. Borna disease was assessed in 72% (13/18) of the BDV-infected rats and only 33% (5/15) of the BDV-infected MAb-treated rats. By day 30 p.i. 80% of the BDV-infected rats (12/15) and 50% of the BDV-infected MAb treated rats (5/10) displayed signs of Borna disease. None of the uninfected control rats showed signs of disease.

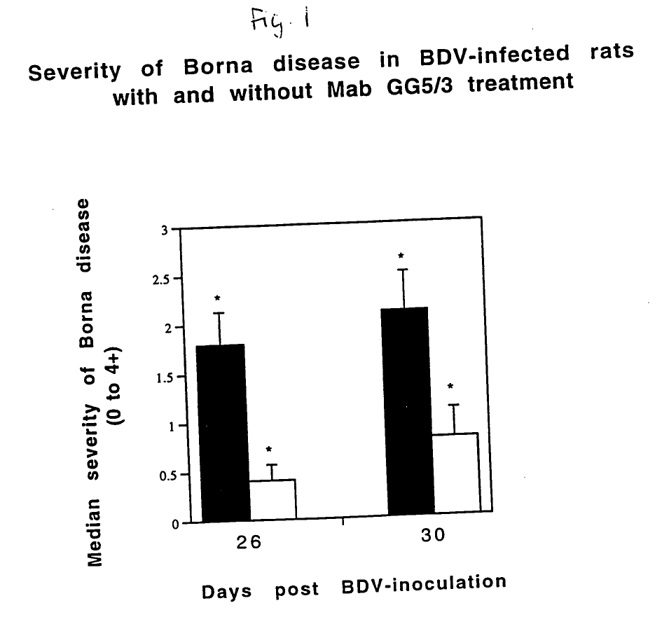

Reduction in Severity of Borna Disease Following Anti-Alpha-4 Integrin Treatment (FIG. 1)

By day 26 p.i., anti-alpha-4 integrin MAb treatment was associated with a reduction in the severity of Borna disease. The severity of disease decreased from 1.8+ (range: 0 to 4+; SEM 0.329; n=18) in the BDV-infected rats to 0.4+ (range: 0 to 2+; SEM 0.163; n=15) in the BDV-infected/MAb treated group, (p<0.05). On day 26 p.i., the majority of BDV-infected rats showed signs of Borna disease while the majority of treated infected rats were symptom free. Not only was the overall incidence of Borna disease reduced in association with anti-alpha-4 integrin treatment but there was also reduction in disease severity. By day 30 p.i. anti-alpha-4 integrin MAb treatment continued to protect BDV-infected rats from developing severe Borna disease. The severity of disease decreased from 2.1+ (range: 1+ to 4+; SEM 0.4; n=14) in the BDV-infected group to 0.8+ (range: 0 to 2+;SEM 0.3; n=10) in the BDV-infected/MAb treated group, (p<0.05).

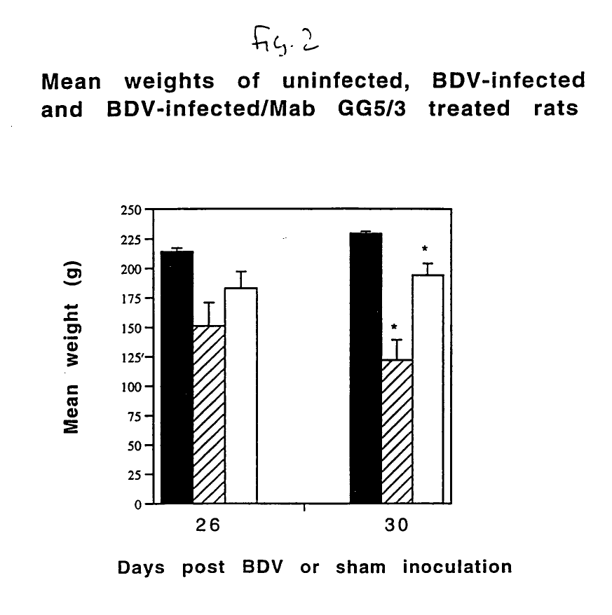

Reduction in Body Weight Loss Following Anti-Alpha-4 Integrin MAb Treatment (FIG. 2)

Body wight loss in BDV-infected rats has been reported as a measure of disease progression (30,31). On day 26 p.i. there was no significant difference between the BDV-infected rat's mean weight of 151 g (range: 114 g to 180 g; SEM 20; n=3) and BDV-infected/MAb-treated rat's mean weight of 183 g (range: 136 g to 211 g; SEM 14; n=5), (p<0.2). However, by day 30 p.i., a significant effect of anti-alpha-4 integrin treatment in limiting BDV-induced weight loss was observed. The BDV-infected group had a mean weight of 122 g (range: 96 g to 155 g; SEM 17; n=3) compared to a mean weight of 194⁹ (range: 164 g to 222 g; SEM 10; n=5) in the BDV-infected/MAb-treated group, (p<0.05). During these time points the uninfected control rats continued to gain weight with mean weights of 214 g (n=2) on day 26 p.i. and 229 g (n=2) on day 30 p.i.

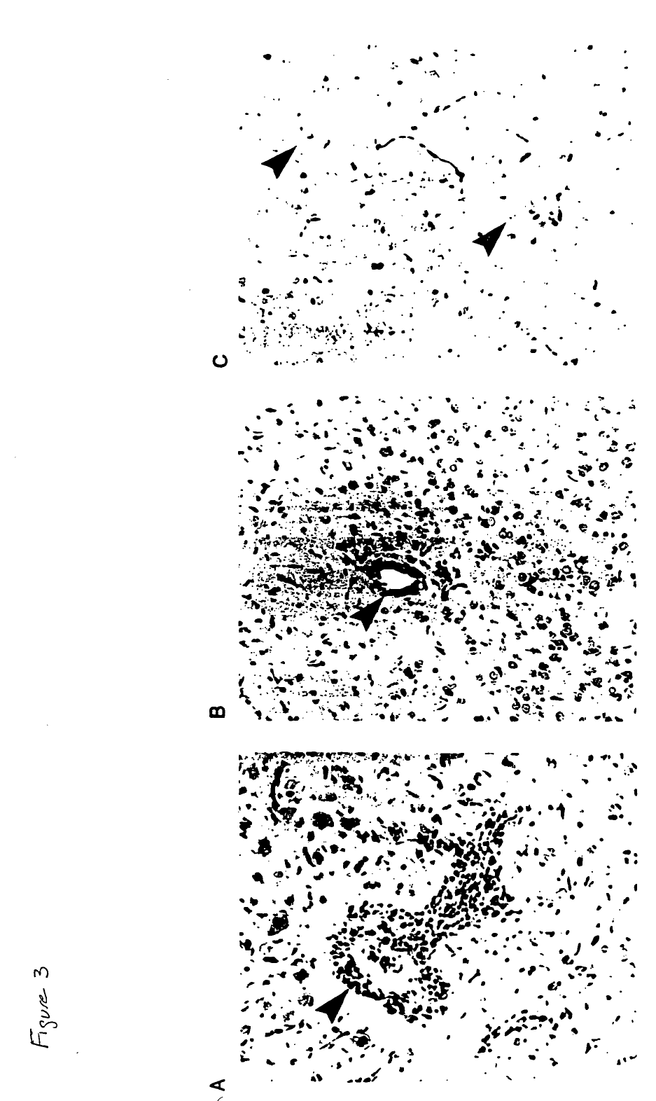

Reduction in the Severity of Encephalitis Following Anti-Alpha-4 Integrin MAb Treatment (FIG. 3)

The degree of encephalitis was rated by microscopic examination of hematoxylin and eosin stained sections of paraffin embedded brain tissue. On day 26 p.i. the BDV-infected rats had a mean encephalitis score of 2.7+ (range: 2+ to 3+; SEM 0.33; n=5) compared to a much reduced rating of 1.2+ (range: 1+ to 2+;SEM 0.2;n=3) in the BDV-infected MAb treated group, (p<0.05) (data not shown). By day 30 the mean severity of encephalitis in the BDV-infected rats increased to 3.3+ (range; 3+ to 4+; SEM 0.33; n=3) FIG. 3A, whereas the mean severity of encephalitis in the BDV-infected/MAb treated rats remained unchanged at 1.2+ (range;1+to 2+;SEM 0.2;n=5) (FIG. 3B). p<0.05). None of the uninfected control rats showed evidence of encephalitis (FIG. 3C).

Viral Titer and BDV Protein Distribution

A comparison of viral titers with and without anti-alpha-4 integrin MAb treatment showed that the reduction in encephalitis did not effect production of infectious BDV, as no statistically significant differences in viral titer were seen between the two groups of rats. On day 26 p.i. a mean of 3.77×10⁴ tissue culture infectious dose fifty (TCID₅₀) of BDV was detected in the brains of the BDV-infected rats as compared to a mean titer of 1.2×10⁴ TCID₅₀ in the BDV-infected/MAb-treated rats, (p<0.57). Likewise, on day 30 p.i. a mean of 1.5×10⁴ TCID₅₀ of BDV was detected in the brain of the BDV-infected rats as compared to a mean of 1.3×10⁴ TCID₅₀ in the BDV-infected MAb treated rats, (p<0.79). Finally, no qualitative differences in viral antigen distribution were observed in the brains of BDV-infected/MAb treated rats.

The data show that despite the remarkable difference in the degree of encephalitis between BDV-infected and BDV-infected/MAb treated rats, viral distribution and infectious virus titers were equivalent in the brains of both groups of rats. Thus, the lack of a destructive encephalitic response did not result in elevated BDV replication in the brain. These data indicate that, the present treatment regime blocks the immunopathological immune response to viral encephalitis without causing enhanced virus replication.

Although the foregoing invention has been described in detail for purposes of clarity of understanding, it will be obvious that certain modifications may be practiced within the scope of the appended claims. All publications and patent documents cited above are hereby incorporated by reference in their entirety for all purposes to the same extent as if each were so individually denoted.

What is claimed is:

1. A method of treating viral encephalitis in a patient, comprising administering to the patient an effect amount of an agent that inhibits binding of leukocytes to brain endothelial cells via leukocyte surface antigen alpha-4 integrin.

2. The method of claim 1, wherein the agent is administered to the patient after viral infection.

3. The method of claim 2, wherein the patient is asymptomatic.

4. The method of claim 2, wherein the patient shows symptoms of encephalitis.

5. The method of claim 1, wherein the agent is administered prophylactically to a patient at risk of infection by a virus causing encephalitis.

6. The method of claim 1, wherein the virus is a herpes virus or an arbovirus.

7. The method of claim 1, further comprising monitoring the patient for symptoms of encephalitis.

8. The method of claim 1, wherein the agent specifically binds to the alpha-4 as a subunit of VLA-4.

9. The method of claim 8, wherein the agent is an antibody.

10. The method of claim 9, wherein the antibody is a Fab fragment.

11. The method of claim 8, wherein the agent binds to an epitope of the alpha-4 subunit formed by association with a beta-1 subunit in an alpha-4 beta-1 complex and lacking in an alpha-4 beta-7 complex.

12. The method of claim 9, wherein the antibody is a humanized antibody.

13. The method of claim 12, wherein the humanized antibody is a humanized form of the mouse 21.6 antibody characterized by a light chain variable domain designated SEQ. ID. No. 1 and a heavy chain variable domain designated SEQ. ID. No. 2.

14. The method of claim 1, further comprising administering an antiviral agent to the patient.

15. The method of claim 1, further comprising administering an antiinflammatory agent to the patient.

16. The method of claim 1, wherein the agent is formulated with a carrier as a pharmaceutical composition.

17. The method of claim 1, wherein the patient is a pediatric patient.


SEQUENCE LISTING (5 sequences)

SEQ ID NO: 1 - protein, 106 aa [sequence omitted]

SEQ ID NO: 2 - protein, 123 aa [sequence omitted]

SEQ ID NO: 3 - protein, 15 aa
  Modified-site 1..6: /product= “OTHER” /note= “Xaa at positions 1-6 may be present or absent, with the proviso that the entire peptide is not more than 11 amino acids”
  Modified-site 7: /product= “OTHER” /note= “Xaa = Tyr or Phe”
  Modified-site 8: /product= “OTHER” /note= “Xaa = Gly or Glu”
  Modified-site 10..15: /product= “OTHER” /note= “Xaa at positions 10-15 may be present or absent, with the proviso that the entire peptide is not more than 11 amino acids”
XXXXXXXXXXXXXXX

SEQ ID NO: 4 - protein, 30 aa
  Modified-site 1..6: /product= “OTHER” /note= “Xaa at positions 1-6 may be present or absent, with the proviso that the entire peptide is not more than 11 amino acids”
  Modified-site 7: /product= “OTHER” /note= “Xaa = Tyr or Phe”
  Modified-site 8: /product= “OTHER” /note= “Xaa = Gly or Glu”
  Modified-site 10..15: /product= “OTHER” /note= “Xaa at positions 10-15 may be present or absent, with the proviso that the entire peptide is not more than 11 amino acids”
  Modified-site 16..21: /product= “OTHER” /note= “Xaa at positions 16-21 may be present or absent, with the proviso that the entire peptide is not more than 11 amino acids”
  Modified-site 22: /product= “OTHER” /note= “Xaa = Tyr or Phe”
  Modified-site 23: /product= “OTHER” /note= “Xaa = Gly or Glu”
  Modified-site 25..30: /product= “OTHER” /note= “Xaa at positions 25-30 may be present or absent, with the proviso that the entire peptide is not more than 11 amino acids”
XXXXXXXXXXXXXXXXXXXXXXXXXXXXXX

SEQ ID NO: 5 - protein, 18 aa
  Modified-site 1..7: /product= “OTHER” /note= “Xaa at positions 1-7 may be present or absent, with the proviso that the entire peptide is not more than 9 amino acids”
  Modified-site 12..18: /product= “OTHER” /note= “Xaa at positions 12-18 may be present or absent, with the proviso that the entire peptide is not more than 9 amino acids”
XXXXXXXPVSFXXXXXXX